(19) United States
(12) Patent Application Publication
(10) Pub. No.: US 2021/0369398 A1
(43) Pub. Date: Dec. 2, 2021

(54) MICROSCOPE SHIELD SYSTEMS, APPARATUSES, AND METHODS

(71) Applicant: EndoPower, LLC, Fort Worth, TX (US)

(72) Inventors: Christopher Yelton, Fort Worth, TX (US); Jason Deblinger, New York, NY (US)

(21) Appl. No.: 17/323,796

(22) Filed: May 18, 2021

Related U.S. Application Data
(60) Provisional application No. 63/030,405, filed on May 27, 2020.

(51) Int. Cl. A61B 90/40; G02B 21/24

(52) U.S. Cl. CPC A61B 90/40; G02B 21/24

(57) ABSTRACT

A microscope shield apparatus may include a physical barrier configured to attach to an operating microscope. The physical barrier may be positioned between a patient and a user while the operating microscope is in use. A method for making a shield apparatus may include forming a physical barrier and forming an aperture within the physical barrier, where the aperture enables attachment of the physical barrier to an operating microscope.

### CROSS-REFERENCE TO RELATED APPLICATIONS

This patent application claims priority to, and the benefit of, U.S. Provisional Patent Application No. 63/030,405 filed on May 27, 2020, and entitled “Microscope Shield Systems, Apparatuses, and Methods,” the contents of which are incorporated by reference herein.

### FIELD OF THE DISCLOSURE

This disclosure is generally related to the field of barriers and shielding and, in particular, to microscope shield systems, apparatuses, and methods.

### BACKGROUND

Microscopes may be used in in medicine, dentistry, veterinary medicine, and other sciences. They may also be used for different procedures including clinical examinations, treatments, and surgeries. Personal protective equipment may protect clinicians and microscope users from pathogens, germs, aerosolized fluids, and other matter from the tissues of a patient and/or other harmful chemicals, substances, and other matter. Existing types of personal protective equipment include masks, face shields, gloves, and other types of wearable protective barriers. Practitioners typically practice great diligence in ensuring that proper personal protective equipment is worn. However, no protective system is absolute. Microscope users may be vulnerable to pathogens, germs, aerosolized fluids, and other matter from the tissues of a patient and other harmful chemicals, substances, and other matter even while employing currently available personal protective equipment.

Also, many processes, procedures, and surgeries involve high revolution instruments and tools, such as drills. During operation there is a risk that these tools may separate and cause projectiles to fly at random angles through the air. These projectiles can potentially cause harm to practitioners or others in the surrounding area. Protection for microscope users is desirable to provide safe clinical conditions.

### SUMMARY

Described herein are microscopy shield systems, apparatuses, and methods that may overcome at least one of the disadvantages associated with clinical microscope use. In an embodiment, a microscope shield apparatus includes a physical barrier configured to attach to an operating microscope, where the physical barrier is positioned between a patient and a user while the operating microscope is in use.

In some embodiments, the physical barrier includes an aperture defined therein, wherein a portion of the operating microscope protrudes through the aperture when the aperture is attached to the microscope. In some embodiments, the physical barrier is planar and has an oval shape. In some embodiments, the oval shape is 20 inches long along a major axis and 16 inches long along a minor axis. In some embodiments, the physical barrier is planar and has a circle shape. In some embodiments, the circle shape has a diameter of 16 inches. In some embodiments, the physical barrier has a capsule shape. In some embodiments, the capsule shape is 20 inches long and 16 inches wide and includes 8 inches of straight edges on both sides of the capsule shape.

In some embodiments, the apparatus includes a fastener configured to attach to the operating microscope and configured to support the physical barrier while attached to the operating microscope. In some embodiments, the fastener includes a friction fit fastener, a zip tie, an elastic band, a screw fastener, another type of fastener, or any combination thereof. In some embodiments, the physical barrier is impervious to pathogens, germs, and biological tissue. In some embodiments, the physical barrier is impervious to chemical substances. In some embodiments, the physical barrier is strong enough to repel a projectile that becomes separated from a revolution instrument during use of the revolution instrument without breaking or cracking. In some embodiments, the physical barrier includes a transparent material that allows visualization of the patient by the microscope user. In some embodiments, the transparent material includes plastic, glass, or polycarbonate.

In an embodiment, a method for making a shield apparatus includes forming a physical barrier. The method further includes forming an aperture within the physical barrier, wherein the aperture enables attachment of the physical barrier to an operating microscope. In some embodiments, forming the physical barrier is performed through a milling operation, a cutting operation, a laser cutting operation, a routing operation, a water jetting operation, a form injection operation, or a combination thereof.

In an embodiment, a shield system includes a physical barrier having an aperture defined therein, where the aperture is configured to receive a portion of an operating microscope therethrough. The system further includes a fastener configured to attach to the operating microscope and to support the physical barrier relative to the operating microscope when attached.

In some embodiments, a size of the physical barrier is sufficient to block all lines of sight between a patient's face and a user's face while the user is viewing the patient's mouth through the microscope. In some embodiments, the physical barrier includes a transparent material that allows visualization of the patient by the microscope user.

### BRIEF DESCRIPTION OF THE DRAWINGS

FIG. 1 is a perspective drawing depicting an embodiment of a microscope shield apparatus in use.

FIG. 2 is a perspective drawing depicting an embodiment of a microscope shield apparatus repelling materials.

FIG. 3 is a schematic diagram depicting an embodiment of an oval shaped microscope shield apparatus.

FIG. 4 is a schematic diagram depicting an embodiment of a circular microscope shield apparatus.

FIG. 5 is a schematic diagram depicting an embodiment of a capsule shaped microscope shield apparatus.

FIG. 6 is flow chart depicting an embodiment of method of forming a microscope shield apparatus.

While the disclosure is susceptible to various modifications and alternative forms, specific embodiments have been shown by way of example in the drawings and will be described in detail herein. However, it should be understood that the disclosure is not intended to be limited to the particular forms disclosed. Rather, the intention is to cover all modifications, equivalents and alternatives falling within the scope of the disclosure.

### DETAILED DESCRIPTION

Disclosed herein is a nonporous physical object attached to an operating microscope that provides a barrier between a microscope user and a source of pathogens, germs, aerosolized fluids, and other matter from the tissues of a patient or other subject matter and other harmful chemicals, substances, and other matter. The barrier may increase the level of safety to microscope users and lower the opportunity for contamination, infection, or other injury.

The barrier may protect vulnerable areas of the microscope user such as the face and eyes, where introduction of pathogens, germs, aerosolized fluids, and other matter from the tissues of a patient may lead to illness or disease. The barrier, also referred to herein as a microscope shield, may include a clear material which allows the microscope user to visualize the patient much in the same way that they would be able to if the microscope shield were not in place. The microscope shield may also provide a barrier to protect the patient from pathogens, germs, or aerosolized fluids that may result from the microscope user during respiration, vocalization, sneezing, or coughing.

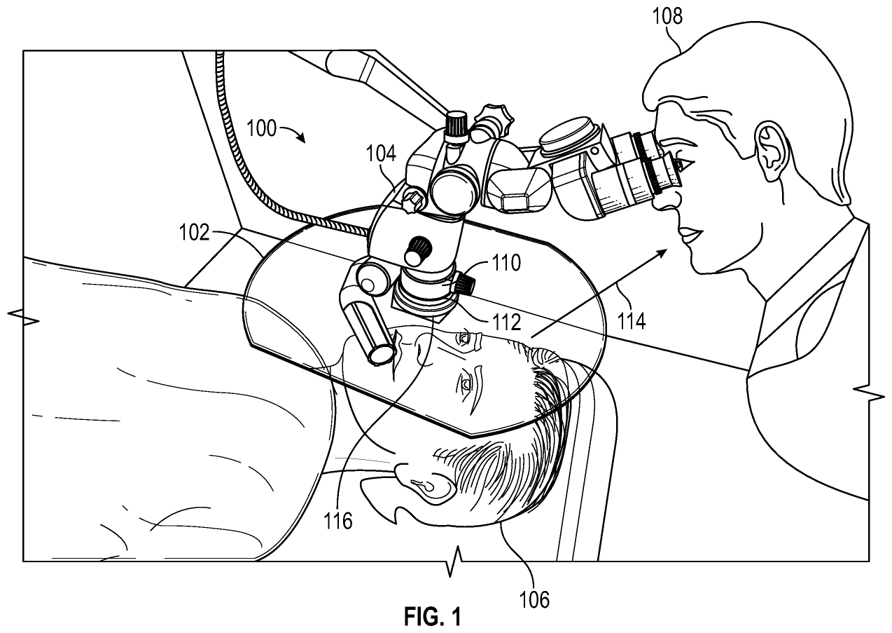

Referring to FIG. 1, an embodiment of a microscope shield apparatus 100 is depicted. The apparatus 100 may attach to an operating microscope 104. In FIG. 1, the operating microscope 104 is depicted in use by a user 108 in examining a patient 106. The user 108 may be a medical practitioner including a medical doctor, a dentist, a veterinarian, an assistant thereof, or any other person who may use the microscope 104 for examining the patient 106. Although the patient 106 is depicted through a silhouette drawing of a human head, in practice the patient 106 may, in some circumstances, be an animal, such as a pet animal or wild or tamed animal that are being treated.

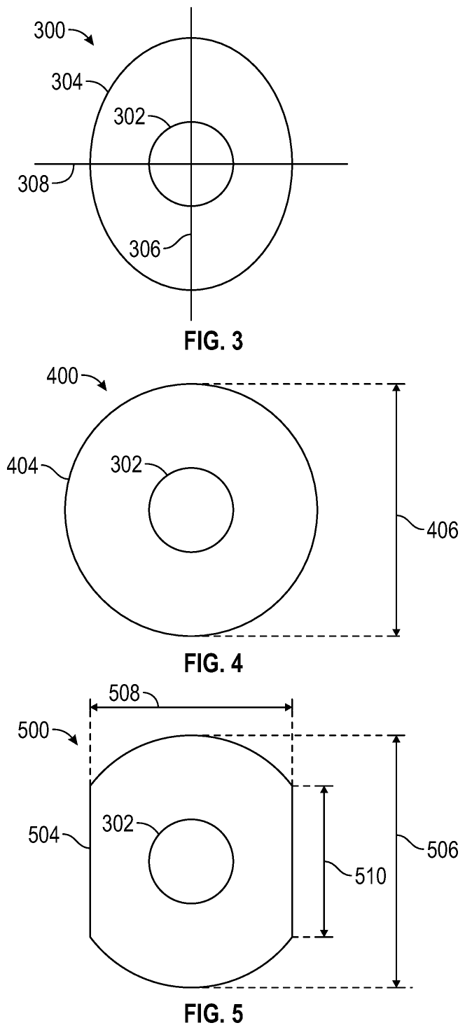

The apparatus 100 may include a physical barrier 102 configured to attach to the operating microscope 104. The physical barrier 102 may include an aperture (described in FIGS. 3-5) defined in the center thereof. A portion 110 of the operating microscope 104 may protrude through the aperture when the physical barrier 102 is attached to the operating microscope 104. Although in FIG. 1, the portion 110 appears to correspond to a lens cylinder of the operating microscope 104, other portions or locations may extend through the physical barrier 102. A size of the physical barrier 102 may be sufficient to block a line of sight 114 between a face of the patient 106 and a face of the user 108 while the user 108 is viewing the patient 106 through the operating microscope 104 with the face of the patient 106 under a lens 116 of the microscope 104 as shown. In other words, the line of sight 114, when traveling from any point on the face of the patient 106 to any point on the face of the user 108 passes through the physical barrier 102. In that way, the physical barrier 102 may block all lines of sight between the faces.

The physical barrier 102 may includes a transparent material that allows visualization of the patient 106 by the microscope user 108. For example, for example, the transparent material may include a plastic, a glass, a polycarbonate, or another type of transparent structural material. A fastener 112 may attach the physical barrier 102 to the operating microscope 104 and may support the physical barrier 102. For example, the fastener 112 may sit below the physical barrier 102 and clasp to the operating microscope 104 via a friction fit. Thus, the fastener 112 may prevent the physical barrier 102 from falling from the operating microscope 104. Other configurations are possible. The fastener 112 may include a friction fit fastener, a zip tie, an elastic band, a screw fastener, another type of fastener, or any combination thereof.

An advantage of the apparatus 100 is that it may provide shielding, protection, and guard the user 108 of the microscope via the physical barrier 102, which may impede access to vulnerable areas of the user 108 such as the face and eyes from pathogens, germs, aerosolized fluids, and other matter from the tissues of the patient 106 and other harmful chemicals, substances, and other matter. The physical barrier 102 may be made of a transparent or clear material allowing visualization of the patient. Other advantages may exist.

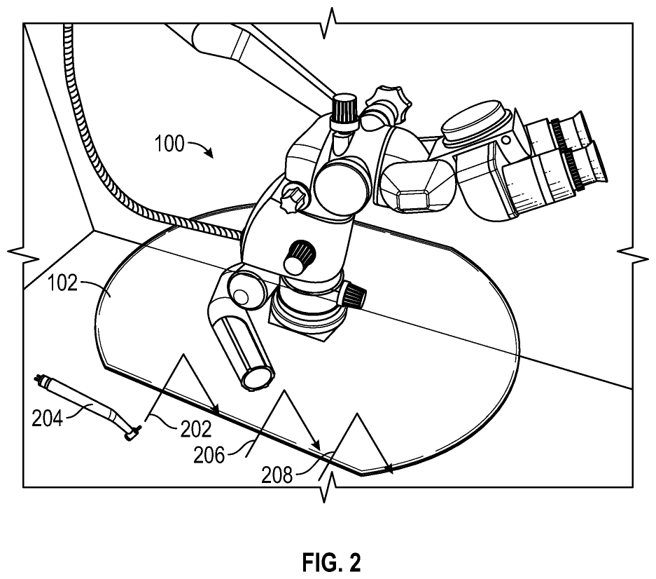

Referring to FIG. 2, a perspective drawing depicting an embodiment of a microscope shield apparatus 100 repelling various materials. For example, the physical barrier 102 may be impervious to biological matter 208, such as pathogens, germs, and biological tissue. Further the physical barrier 102 may be impervious to chemical substances 206, such as chemical treatment materials, antiseptic materials, soaps, detergents, acids, etc. As used herein, the term “impervious” means that the material of the physical barrier 102 does not permit a fluid (liquid, gas, or aerosol) to immediately pass through it. It should be noted that some chemicals may react over time with the physical barrier 102, but that the physical barrier 102 may nevertheless be impervious to those chemicals at least temporarily until its structure breaks down. The physical barrier 102 may be strong enough to repel a projectile 202 that may become separated from a revolution instrument 204 during use of the revolution instrument 204 without breaking or cracking. As used herein, the term “strong” means a combination of tensile strength, compressive strength, shear strength, torsional strength, and other types of physical structure strengths sufficient to deflect projectiles launched from typical rotary medical tools without breaking or cracking.

The disclosed microscope shield apparatus 100 may be of various shapes including circular, ovoid, capsule, rectangular, or any number of different shapes including amorphous shapes. Some non-limiting examples of these shapes are provided in FIGS. 3-5. The dimensions of the microscope shield apparatus 100 may vary in length, width, and thickness. Further, the microscope shield may be flat, concave, or convex, depending on a particular application or potion. The microscope shield's shapes, dimensions, contour, presence of aperture and its size, attachment apparatus and location may vary depending on the design of a microscope and based on a manufacturer and a model for which it may be be intended.

Referring to FIG. 3, an embodiment of an oval microscope shield apparatus 300 is depicted. The shield apparatus 300 may have an oval shape 304 having an aperture 302 defined therein to enable attachment of the shield apparatus 300 to an operating microscope. For example, a portion of an operating microscope may protrude through the aperture 302 when the shield apparatus 300 is attached to the microscope. The aperture 302 may be circular and have a diameter of about 2.6 inches. Other shapes and dimensions of the aperture 302 are possible depending on the microscope to be used.

The apparatus 300 may be planar, concave, or convex. The oval shape 304 may be defined in a plan view of the apparatus 300. As used herein, the term “oval” may include ellipsoids, a straight edged oval, and any other type of skewed circular shape. Although many dimensions may be possible depending on application, the oval shape 304 may be about 20 inches long along a major axis 306 and about 16 inches long along a minor axis 308. The size may be sufficient to block all lines of sight between a patient's face and a microscope user's face, while the microscope user examiners the patient under the microscope.

Referring to FIG. 4, an embodiment of a circular microscope shield apparatus 400 is depicted. As with the apparatus 300, the apparatus 400 may include a circular aperture 302 defined in a center thereof. As before, the aperture 302 may be circular and have a diameter of about 2.6 inches with other shapes and sizes also being possible. The apparatus 400 may have a circular shape 404. Although other dimensions may be possible, the circular shape 404 may have a diameter 406 of about 16 inches. As before, the size may be sufficient to block all lines of sight between a patient's face and a microscope user's face, while the microscope user examiners the patient under the microscope.

Referring to FIG. 5, an embodiment of a capsule shaped microscope shield apparatus 500 is depicted. As with the apparatuses 300, 400 the apparatus 500 may include an aperture 302 defined in a center thereof. As before, the aperture 302 may be circular and have a diameter of about 2.6 inches with other shapes and sizes also being possible. The apparatus 500 may have a capsule shape 504 with a rounded top and bottom and straight sides. The capsule shape 504 may have a length 506 of about 20 inches and a width 508 of about 16 inches. The capsule shape 504 may also include a length 510 of about 8 inches of straight edges on both sides of the capsule shape 504. As before, the size may be sufficient to block all lines of sight between a patient's face and a microscope user's face, while the microscope user examiners the patient under the microscope.

In a particular application, a capsule shaped shield apparatus was developed having a capsule shape with a length of 14.625 inches and a width of 18 inches. The apparatus further included 10 inches of straight edges along the sides. The apparatus was computer numerical control (“CNC”) machined from ⅛-inch clear polycarbonate material with a 2.6-inch diameter aperture in the center. Further, the outside edge was smoothed and rounded.

Manufacturing the any of the apparatuses 100, 300, 400, 500 may take place through by milling, cutting, laser cutting, routing, water jetting, form injection, or other form of manufacturing of a clear and transparent material into one of many various and possible shapes including circular, ovoid, capsule, rectangular, or any number of different shapes including amorphous shapes with varying dimensions of length, width, and thickness and contain an aperture or connection to which the microscope shield can attach to the microscope. The shapes, dimensions, aperture size and location of the aperture may vary depending on the design of the microscope for which it will be intended.

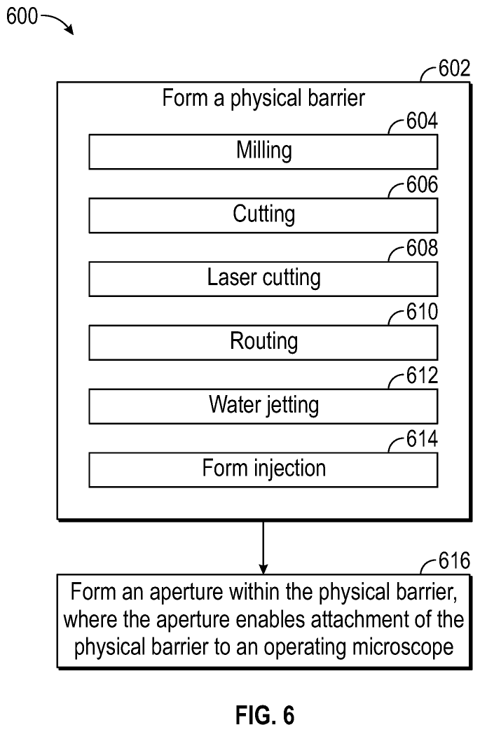

FIG. 6 is flow chart depicting an embodiment of method of forming a microscope shield apparatus. The method 600 may include forming a physical barrier, at 602. The physical barrier may be formed from a planar, concave, or convex clear material, such as plastic, glass, or polycarbonate. One or more processes may be used to form the physical barrier, such as milling, at 604, cutting, at 606, laser cutting, at 608, routing, at 610, water jetting, at 612, and form injection, at 614.

The method 600 may further include forming an aperture within the physical barrier, where the aperture enables attachment of the physical barrier to an operating microscope, at 616. For example, the physical barrier 102 may be formed with an aperture, such as the aperture 302, formed therein to enable attachment of the physical barrier 102 to the operating microscope 104.

Although various embodiments have been shown and described, the present disclosure is not so limited and will be understood to include all such modifications and variations as would be apparent to one skilled in the art.

1. A microscope shield apparatus comprising:
a physical barrier configured to attach to an operating microscope, wherein the physical barrier includes an aperture defined therein, wherein a portion of the operating microscope protrudes through the aperture when the aperture is attached to the microscope

2. The apparatus of claim 1, wherein the physical barrier is positioned between a patient and a user while the operating microscope is in use.

3. The apparatus of claim 1, wherein the physical barrier is planar and has an oval shape.

4. The apparatus of claim 3, wherein the oval shape is 20 inches long along a major axis and 16 inches long along a minor axis.

5. The apparatus of claim 1, wherein the physical barrier is planar and has a circle shape.

6. The apparatus of claim 5, wherein the circle shape has a diameter of 16 inches

7. The apparatus of claim 1, wherein the physical barrier has a capsule shape.

8. The apparatus of claim 7, wherein the capsule shape is 20 inches long and 16 inches wide and includes 8 inches of straight edges on both sides of the capsule shape.

9. The apparatus of claim 1, further comprising:
a fastener configured to attach to the operating microscope and configured to support the physical barrier while attached to the operating microscope.

10. The apparatus of claim 9, wherein the fastener includes a friction fit fastener, a zip tie, an elastic band, a screw fastener, another type of fastener, or any combination thereof.

11. The apparatus of claim 1, wherein the physical barrier is impervious to pathogens, germs, and biological tissue.

12. The apparatus of claim 1, wherein the physical barrier is impervious to chemical substances.

13. The apparatus of claim 1, wherein the physical barrier is strong enough to repel a projectile that becomes separated from a revolution instrument during use of the revolution instrument without breaking or cracking.

14. The apparatus of claim 1, wherein the physical barrier includes a transparent material that allows visualization of the patient by the microscope user.

15. The apparatus of claim 14, wherein the transparent material includes plastic, glass, or polycarbonate.

16. A method for making a shield apparatus comprising:
forming a physical barrier; and
forming an aperture within the physical barrier, wherein the aperture enables attachment of the physical barrier to an operating microscope.

17. The method of claim 16, wherein forming the physical barrier is performed through a milling operation, a cutting operation, a laser cutting operation, a routing operation, a water jetting operation, a form injection operation, or a combination thereof.

18. A shield system comprising:
a physical barrier having an aperture defined therein, wherein the aperture is configured to receive a portion of an operating microscope therethrough; and
a fastener configured to attach to the operating microscope and to support the physical barrier relative to the operating microscope when attached.

19. The shield system of claim 18, wherein a size of the physical barrier is sufficient to block all lines of sight between a patient's face and a user's face while the user is viewing a mouth of the patient through the microscope.

20. The shield system of claim 18, wherein the physical barrier includes a transparent material that allows visualization of a patient by a microscope user.
